(19) United States
(12) Patent Application Publication
(10) Pub. No.: US 2019/0183603 A1
(43) Pub. Date: Jun. 20, 2019

(54) MEDICAL HEADLAMP AND CAMERA SYSTEM

(71) Applicant: RIVERPOINT MEDICAL, LLC, PORTLAND, OR (US)

(72) Inventor: JOHN THOMAS FERGUSON, PORTLAND, OR (US)

(73) Assignee: RIVERPOINT MEDICAL, LLC, PORTLAND, OR (US)

(21) Appl. No.: 16/273,568

(22) Filed: Feb. 12, 2019

Related U.S. Application Data
(63) Continuation-in-part of application No. 14/510,364, filed on Oct. 9, 2014, now Pat. No. 10,258,411.
(60) Provisional application No. 61/981,695, filed on Apr. 18, 2014.

(51) Int. Cl. A61B 90/53; A61B 90/30; A61B 90/00

(52) U.S. Cl. CPC A61B 90/53; A61B 2090/502; A61B 90/361; A61B 90/30

(57) ABSTRACT

A medical headlamp and camera system having an articulated linkage-and-headlamp assembly, including an articulated linkage supporting a headlamp and a video camera, which produces a first video data signal. A headband assembly, supporting the articulated linkage-and-headlamp assembly, and including at least one battery port, supporting a battery, and further including an electrical network, including a microcontroller, that supplies electrical power to the headlamp, in reliance on computations performed by the microcontroller, from the battery and also delivers electrical power from the battery to the video camera. Also, a data compression network, is electrically connected to the video camera and receives the first video data signal and compresses it into a compressed data signal, Finally, a wireless transceiver supported by the headband assembly and electrically connected to the data compression network wirelessly transmits the compressed data signal.

### RELATED APPLICATIONS

This application is a continuation-in-part of application U.S. Ser. No. 14/510,364 filed on Oct. 9, 2014, which claims benefit of provisional application Ser. No. 61/981,695 filed Apr. 18, 2014.

### BACKGROUND

Medical headlamp assemblies having attached video cameras are old. These assemblies, however, tend to be heavy and are tethered by cables to a base station. This potentially interferes with the wearer's freedom of movement and may prove to be a distraction during delicate surgical procedures. For medical headlamp assemblies that must be physically tethered, in order to power the headlamp, little benefit could be gained by equipping the assembly with a wireless, as opposed to a wired, camera or vision system.

Untethered medical headlamp assemblies, having efficient lamps that permit the use of battery packs on the headband, are currently available. Typically, an adjustable linkage attaches the lamp to a headband. Although it might at first seem possible to simply attach an existing wireless video camera to the lamp, so that the camera images the area that is being illuminated, size, mass and power constraints may make this an undesirable solution.

Installing a wireless video camera assembly directly on the lamp adds to the weight of the lamp/camera combination, and results in a requirement for a stiffer linkage, to prevent the lamp/camera from drooping. This is particularly true of the camera includes batteries, for its power. But a stiffer linkage is undesirable as this reduces the ease of adjustment. Also, a bulkier lamp/camera unit may act as a distraction to the wearer, who has some awareness of an element above the lamp, very near his forehead. Finally, a greater mass results in greater inertia when the wearer rotates his head, resulting in an unpleasant sensation during head rotation, and more torque at the location where the linkage holding up the lamp meets the headband. A Wi-FI antenna and a camera battery, at least, are problematic elements to include in the housing with the camera.

Moreover, transmitting raw video over a WI-FI link can consume upwards of 2 watts of power. This means that a complete WI-FI camera system would consume more power than the medical headlamp, thereby requiring over frequent battery swap-outs, and appearing impractical. It is therefore desirable to compress the video signal, prior to transmitting this signal.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

In a first separate aspect, the present invention may take the form of a medical headlamp and camera system having an articulated linkage-and-headlamp assembly, including an articulated linkage supporting a headlamp and a video camera, which produces a first video data signal. A headband assembly, supporting the articulated linkage-and-headlamp assembly, and including at least one battery port, supporting a battery, and further including an electrical network, including a microcontroller, that supplies electrical power to the headlamp, in reliance on computations performed by the microcontroller, from the battery and also delivers electrical power from the battery to the video camera. Also, a data compression network, is electrically connected to the video camera and receives the first video data signal and compresses it into a compressed data signal, Finally, a wireless transceiver supported by the headband assembly and electrically connected to the data compression network wirelessly transmits the compressed data signal.

In a second separate aspect, the present invention may take the form of a medical headlamp and camera system having an articulated linkage-and-headlamp assembly, including an articulated linkage supporting a headlamp and a video camera, which produces a first video data signal. A headband assembly, supporting the articulated linkage-and-headlamp assembly, and including at least one battery port, supporting a battery, and further including an electrical network, including a microcontroller, that supplies electrical power to the headlamp, in reliance on computations performed by the microcontroller, from the battery and also delivers electrical power from the battery to the video camera. Also, a data compression network, is electrically connected to the video camera and receives the first video data signal and compresses it into a compressed data signal, Finally, a wireless transceiver supported by the headband assembly and electrically connected to the data compression network wirelessly transmits the compressed data signal. Also, a wireless transceiver is supported by the headband assembly and electrically connected to the data compression network to wirelessly transmit the compressed data signal. The electrical network processes data representative of the first data signal by detecting a region illuminated by the bezel and eliminating from further processing pixels outside of the illuminated region.

In a third separate aspect, the present invention may take the form of a medical headlamp and camera system, having a headband assembly, that includes a headband, having at least one battery port, supporting a battery, and further including an electrical network, including an integrated circuit. The headband assembly also has an articulated linkage-and-headlamp assembly supported by the headband, and including an articulated linkage supporting a headlamp and an electrically conductive connection from the electrical network to the headlamp powering the headlamp from the electrical network. Also, an image sensor is supported by the headband assembly such that it can be directed to gather imagery from a region illuminated by the headlamp and electrically and communicatively connected to and powered by the electrical network and producing a first video data signal. Further, a data compression network is electrically connected to the video camera, which receives the first video data signal and compresses it into a compressed data signal. In addition, a wireless transceiver is supported by the headband assembly and electrically connected to the data compression network to wirelessly transmit the compressed data signal. Finally, the electrical network processes data representative of the first data signal and controls the headlamp brightness in response thereto.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced drawings. It is intended that the embodiments and figures disclosed herein are to be considered illustrative rather than restrictive.

FIG. 1 is a block diagram of the electrical power and logic of a medical headlamp and video camera headband assembly according a preferred embodiment of the present invention.

FIG. 2 is an isometric side-top view of a medical headlamp assembly according to the present invention, configured to be received onto a user's head.

FIG. 3 is an isometric side-top view of the assembly of FIG. 2, but without the tightness adjustment elements, and with elements extended outwardly, in a plane.

FIG. 4 is a front view of the assembly of FIG. 3.

FIG. 5 is an isometric side-top view of a rigid-flex circuit element of the assembly of FIG. 3.

FIG. 6 is a front view of the rigid-flex circuit element of FIG. 5.

FIG. 7 is a section view of the assembly of FIG. 4, taken along view line 7-7 of FIG. 4.

FIG. 8 is a section view of the assembly of FIG. 4, taken along view line 8-8 of FIG. 4.

FIG. 9 is an isometric side-top view of a medical headlamp assembly, representing an alternative embodiment of the present invention, configured to be received onto a user's head.

### DETAILED DESCRIPTION OF THE PREFERRED EMBODIMENTS

Definitions: A data signal is any signal that represents a stream of data. Transforming a data signal means changing the representation of a data value, for example changing from a signal in which a “1” is represented by 0.34 volts on a data line, to a signal in which a “1” is represented by 2.3 volts or a signal where a “1” is represented by a value of an electromagnetic signal, present in the air. Transforming a data signal also includes transforming the data represented by the data signal by, for example, compressing it or encrypting it.

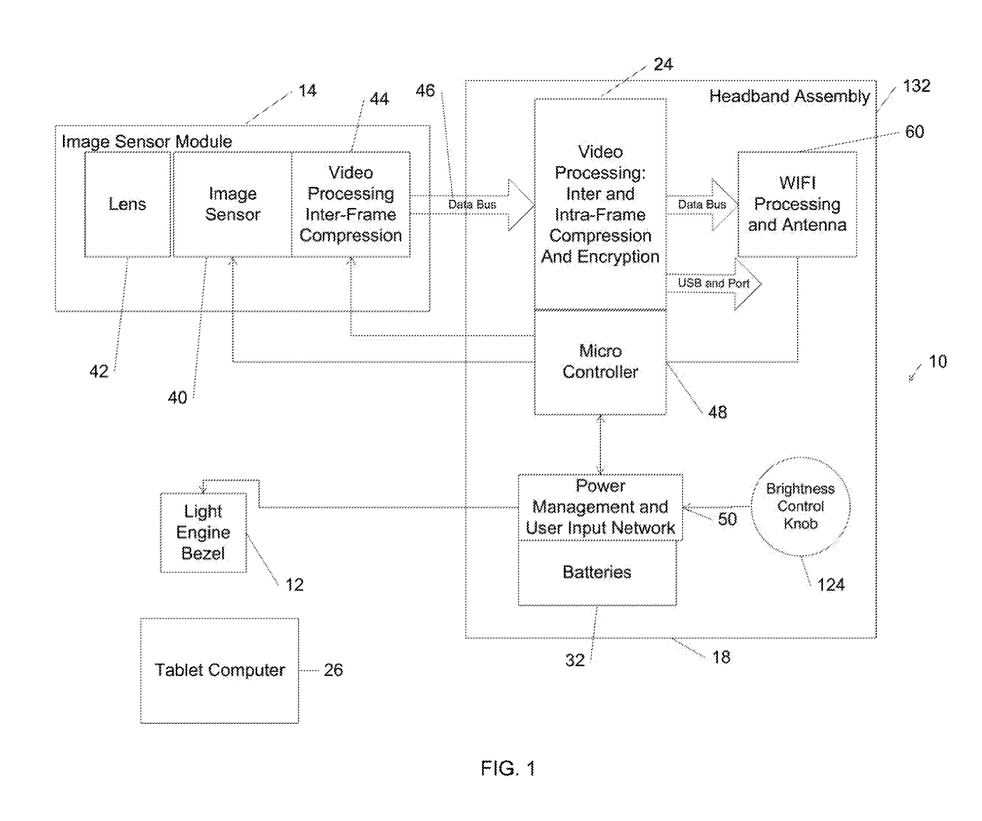

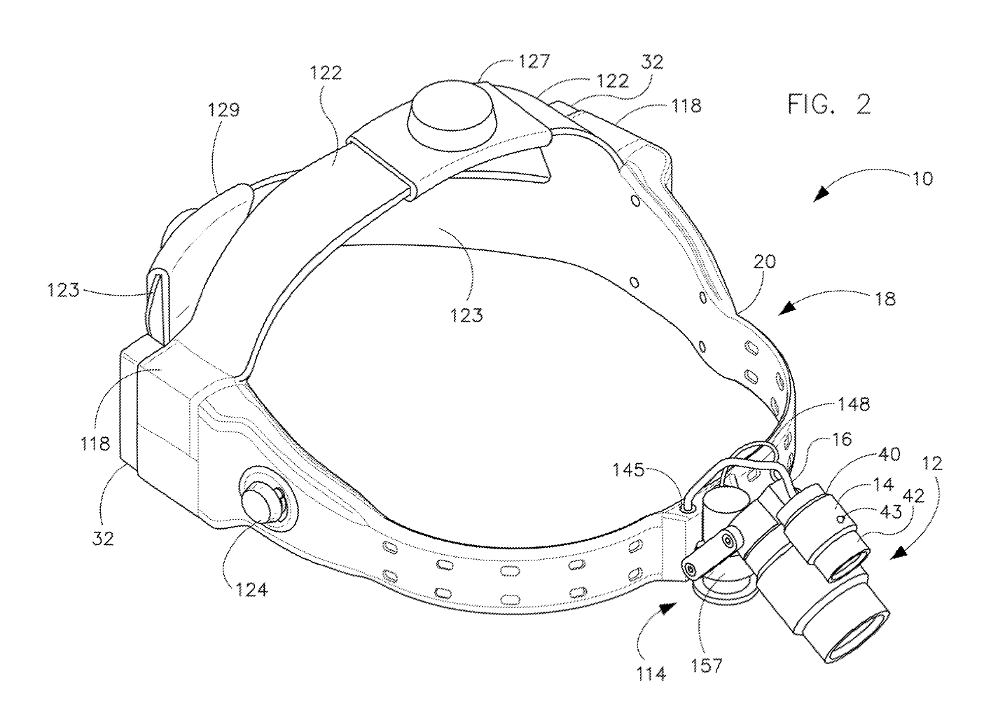

Referring to FIGS. 1 and 2, a preferred embodiment of a medical headlamp and camera system 10 includes a headlamp (also referred to as “light engine bezel”) 12 and an image sensor module (also referred to as “video camera” or simply “camera”) 14. Image data gathered by module 14 is sent over a multi-conductor cable 16 to a headband assembly 18 that includes a headband structure 20 and internal electrical conductors that lead to a principal video processor 24, which processes and wirelessly transmits the image data. A tablet computer 26 communicates with system 10, by way of Wi-Fi Processing and Antenna 60, and is configured to receive the wireless image data, and to display and record it as commanded by a user. Tablet computer 26 is also configured to wirelessly command micro-controller 48, through Wi-Fi unit 60, which is connected to micro-controller 48, to, for example, command it to command the image sensor module 14 to begin recording. In one preferred embodiment, tablet computer 26 may use the same communications route to adjust the brightness of headlamp 12. A pair of battery ports 118, support batteries 32 that power the system, with wire 148 supplying headlamp 12 with electrical power. In one preferred embodiment tablet computer 26 includes a microphone and voice command recognition technology, so that it can be used to relay voice commands to assembly 10.

Image sensor module 14, includes an image sensor 40, a lens stack 42 and a front-end video processor 44. Lens stack 42 sets the field of view and focus distance of module 14. In one preferred embodiment, lens stack 42 is adjustable to set a precise focus length based on the anticipated distance at which the camera 14 will be viewing a medical procedure, and can be set in place, after adjustment by, for example, set screw 43. In a variant of this, differing versions of a front end of the lens stack 42 will be made available, each optimized for a different anticipated viewing distance, so that the user may select the front end that best suits his needs. A surgeon may set the focal length of the lens stack 42 of his assembly 10 based on his arm length.

Image sensor 40 and video processor 44 may collectively take the form of the Aptina AS0260, which includes a system-on-a-chip and has the capability to perform intra-frame data compression, more specifically in accordance with the JPEG (Joint Photographic Experts Group) standard or MJPEG standard. In an alternative embodiment image sensor 40 may take the form of a sensor with an ultra-low energy change sensing mode, so that the camera 14 can draw as little power as possible for an image that is not undergoing significant change but can be commanded to take much higher resolution and higher frame rate imagery as soon as an image change is detected. Another option for image sensor 40 is the AS01402AT, available from On Semiconductor, which maintains a website at www.onsemi.com.

A multi-line data bus 46, of which multi-connector cable 16 (FIG. 2) forms a part, connects the image sensor module 14 to a principal video processor 24. A micro-controller 48 controls the system 10. The principal video processor 24 and the control unit 48, collectively may take the form of an Ambarella A7LW system on a chip (SoC). This unit includes an ARM processor, which serves as control unit 48 and a video processing unit, which serves as principal video processing unit 24, which compresses and encrypts the data, according to an interframe scheme of compression. In one preferred embodiment, the H.264/MPEG-4 AVC standard is used for the data compression and encryption.

Also, within headband assembly 18, a power management network 50, receives power from a battery set 32 and delivers power to the light engine bezel 12, the image sensor module 14 and the power consuming units of the headband assembly 18. A brightness control knob 124 permits a user to adjust the brightness of the bezel 12. In one preferred embodiment, knob 124 also acts as an on/off switch for system 10, so that as knob 124 is rotated to an extreme position in a first direction, a “click” sound is made and the entire system 10 is turned off. When rotated in the opposite direction a similar click sound is made and the system is turned on, with the WIFI unit 60 placed in listen mode, to receive a command from tablet computer 26, for image sensor 40 to begin recording, and bezel 12 to illuminate.

A WI-FI unit 60, including an antenna, broadcasts the data received from processing unit 24. System 10 also includes a USB port, for connection of a dongle or a USB cable.

The object of the data processing scheme implemented in both front end processor 44 and principal processor 24 is to determine and send the most important data that can be fit into the limited bandwidth available (about 100 MBPS or less) that can be used without using so much energy as to become burdensome to the operating room crew, which must swap out batteries that have been depleted. One technique that is used is the detection of the region of the frames that is illuminated by the bezel 12 and delivering only information representative of this region to WI-FI unit to be transmitted. A first order detection scheme tests for a border between bright and dim pixels, or otherwise detects the area illuminated by the bezel 12 and eliminates the pixels on the outside of this area. In one embodiment, this processing is not performed every frame, because the relationship between image sensor 14 and bezel 12 will typically not change very quickly. Rather, in one embodiment, after the headlamp and camera are activated, the processor 24 attempts to detect a spot of light. It is possible that there will be none, because the lamp might not be directed at a surface that is close enough to provide a definite light spot. In one methodology, a line of pixels going through or close to the center of the frame is examined, to see if a set in the middle is brighter than those at the ends. For example, if a set of 20 contiguous pixels are found that are twice as bright as five contiguous pixels at either end of the line, then a spot of light may be considered to be tentatively detected. In another preferred embodiment the center pixels must be 10 times as bright for a tentative light spot detection to be noted. Further processing along other lines of pixels running through or close to the center may be used to confirm or disqualify the tentative detection of a light spot. After that initial light spot detection, subsequent frames of pixels may be used to refine the estimate of light spot location. After that, for each frame only the set of pixels found to be in the light spot are processed and transmitted or saved. In one embodiment, a periodic check is performed, for example every second or every two seconds or every five seconds, to verify that the light spot has not moved in the field of view of the image sensor 14. Further the data compression scheme is optimized for the type of data likely to be encountered during a surgery. In one preferred embodiment the data compression scheme is matched to an expected rate of change of imagery during surgery. In one embodiment, a tablet 26 user can choose a first portion of the camera field of view to be broadcast at a fast data rate, and a second portion of the camera field of view to be broadcast at a slow data rate, slower than the fast data rate. In one embodiment, the user of tablet 26 can superimpose a circle, or other closed form indicator, on the field of view, thereby commanding the imagery within the circle to be broadcast at a faster rate, for example 60 frames per second, 30 frames per second or 24 frames per second, and everything outside of the closed form indicator to be broadcast at a slower data rate, for example 1 frame per second, or 1 frame every 5 seconds. The user direction is sent to WIFI 60, from tablet computer 26.

In one embodiment, every video data file produced by system 10 is labelled as being patient -sensitive medical information, so that it will be safeguarded from disclosure to anybody other than appropriate medical personnel, to aid in compliance with the Health Insurance Portability and Accountability Act (HIPAA) and any other relevant laws, regulations and procedures. Different systems exist for labelling information for protection, and in one embodiment, a user may select the labelling system. In another embodiment, every frame, in the margin, includes a warning that the imagery is patient-sensitive and subject to HIPAA regulations. In one embodiment, the video produced is password protected, with an automatically generated password being automatically provided to a qualified person, such as the surgeon performing the surgery, by a system that safeguards the information from others. In a preferred embodiment a video library is maintained, and every user of the library can log into an account, using a secret password and a user ID, to see only those videos that are assigned to him or her. Those without a password or user ID are prevented from accessing any of the videos. In one embodiment headlamp assembly 10 includes a biometric sensor, such as a fingerprint sensor, to verify the identity of the user, so that he or she can be given access to the video, without error.

In a preferred embodiment, an accelerometer set is placed on the camera 14, bezel 12 or linkage 114. Information from this accelerometer set is sent by WIFI and used to deblur imagery from camera 14, for example if the surgeon shifts his head position and the camera 14 is rapidly moved, the accelerometer yields this information and it is used to deblur the resultant imagery.

In another scheme, the rate of wireless transmission is slowed during periods when there is less movement in the field of view. In one embodiment a gravity sensor (plumb bob) is used to detect instances in which the surgeon has straightened himself, from the typical position during surgery of bending over a surgical theater, and can therefore be presumed to be no longer viewing the surgical theater, so that the bezel 12 may be turned off or dimmed, and the video camera may be turned off. In one embodiment, these power saving devices can be left unused at the user's input to the contrary. In one embodiment an especially low transmit power wireless system is used, to reduce power consumption, take advantage of the nearness of the wireless receiver and avoid interference with other RF equipment in the medical environment. In one embodiment, the device receiving the video signal, such as tablet computer 26, sends a signal back to WIFI unit 60 and therefore micro-controller 48, instructing micro-controller 48 to cause data to be broadcast at a lower signal volume, to arrive at the lowest signal volume that avoids unnecessary signal strength, that could disturb other wireless processes in the hospital. In one embodiment a preset signal is sent periodically from WIFI unit 60 to the tablet computer 26, which determines if the preset signal has arrived correctly. If it has not the tablet computer 26 sends a signal requesting a higher signal intensity. In one embodiment a blue-tooth system is used for transmitting the video signal. In one embodiment a maximum of two watts of power are used to power the bezel 12, the image sensor 14, the processor 24 and the wireless transmitter 60.

In one preferred embodiment a user selectable mode is provided in which the control unit adjusts the current delivered to bezel 12, in response to the brightness of the pixels within the illuminated area, thereby saving electricity when the illuminated circle is brighter than necessary. The user may opt out of this mode, to avoid possible complications. One problem faced by surgeons is reflection from the scalpel and/or other surgical instruments used. Such a reflection may temporarily blind the surgeon and cause a brief interruption in a surgery. Surgical time is extremely valuable, and the surgeon's full attention necessary to good outcomes, so it is very desirable to reduce or eliminate anything that interferes with the surgical process. In one embodiment, when an image sensor pixel is suddenly illuminated much more brightly than it had been before, and particularly if it becomes illuminated more brightly than other pixels within the light spot, an algorithm detects this condition and reduces the volume of light produced by the bezel, in response. For example, in one embodiment if the volume of light in any 10 contiguous camera pixels triples in less than 0.2 seconds and exceeds a set threshold for light from 10 contiguous pixels, the light output of the bezel 12 is reduced.

In another embodiment a user can set a set point, by for example turning the brightness knob, while camera 14 is active, to a place where the amount of light produced appears beneficial, and then give some further indication, for example pressing the knob or another button, to indicate that this level of light return is the desired level. Subsequently, if the light returned from the detected spot exceeds this level, the light output of bezel 12 is reduced by the micro-controller 48, and if the light returned from the detected spot is less than this level, the light output of bezel 12 is increased by the micro-controller 48.

In another embodiment, the light produced by the bezel is reduced if any pixel is driven into saturation, or returns its maximum value, the level of light produced by the bezel is reduced. In one embodiment, the brightest camera pixel is detected for every frame, and compared to the brightest pixel from the previous frame and the brightest pixel from the frame before that. If there is a sudden increase in brightness, in one embodiment a ten-times increase in the brightest pixel level this is attributed to unwanted reflection and the current delivered to the bezel is reduced.

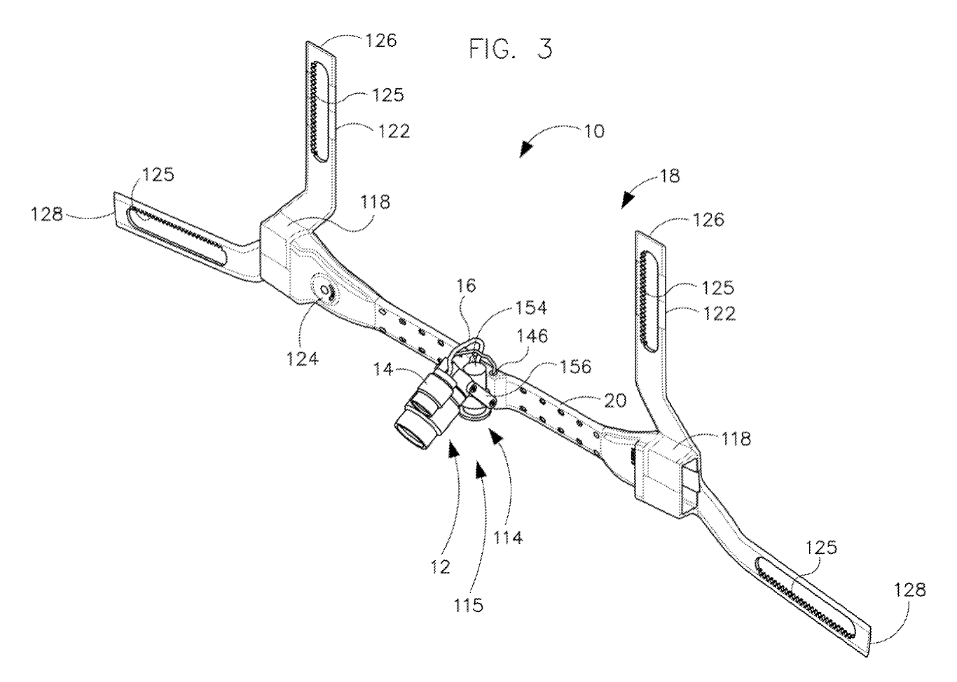

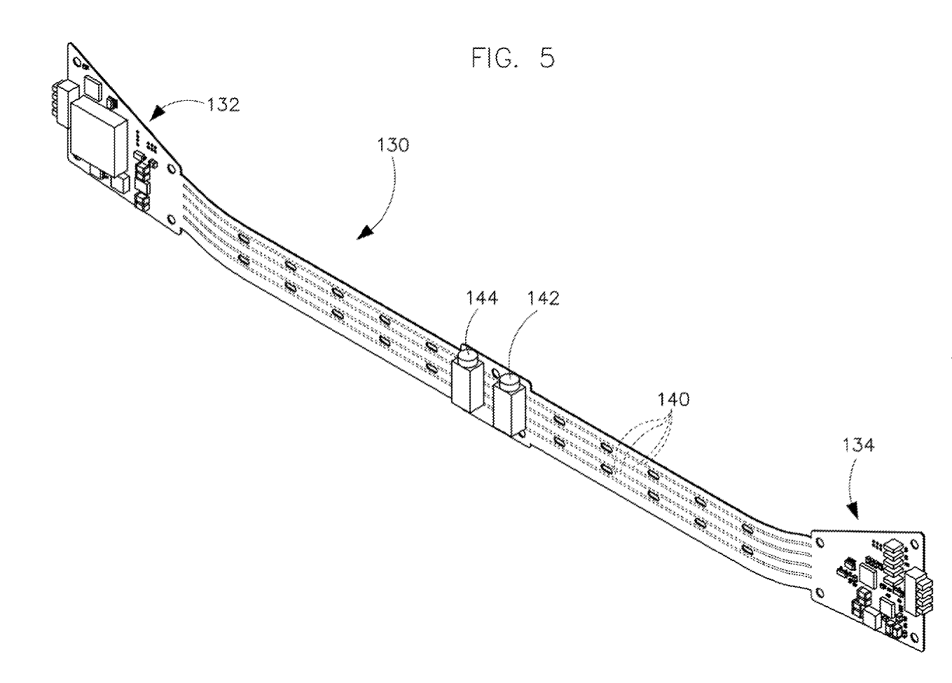

Referring to FIGS. 2 and 3, in physical form, a preferred embodiment of the present invention is a medical headlamp assembly 10, having a light engine bezel 12, a video camera 14, an adjustable bezel support linkage 114, a headband assembly 18, defining a pair of battery sockets 118, bearing batteries 32, each in contact to a rigid-flex circuit insert 130 (FIG. 5). The linkage 114 and light engine bezel 12, collectively constitute an integrated articulated linkage-headlamp and camera assembly 115.

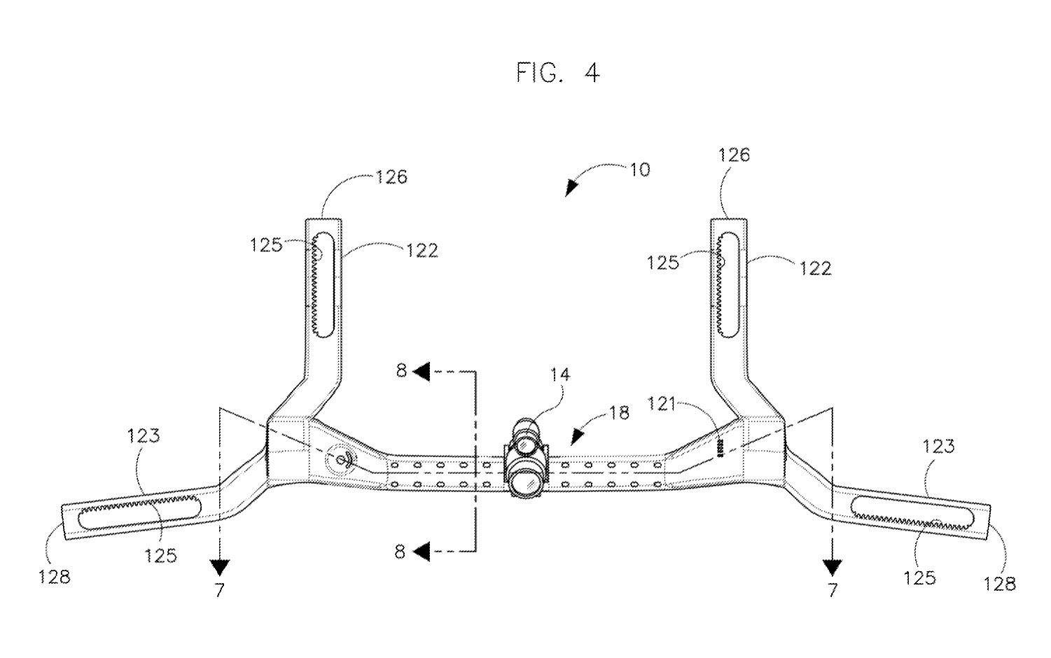

The charge remaining in batteries 32 is indicated by a set of battery charge indicator lights 121 (FIG. 4). A head-top strap 122 and a head-back strap 123 form a part of headband assembly 18. As shown in FIG. 3 straps 122 and 123 are both formed from a pair of arms (126, 128) each having a serrated elongated opening 125. The two arms of the head-top band are drawn together by tightness adjust 127 which engages the serrations to adjust the length of the coupled arms, and the two arms of the head-back strap 123 are drawn together by tightness adjust 129. A brightness control knob 124 is also supported by headband assembly 18.

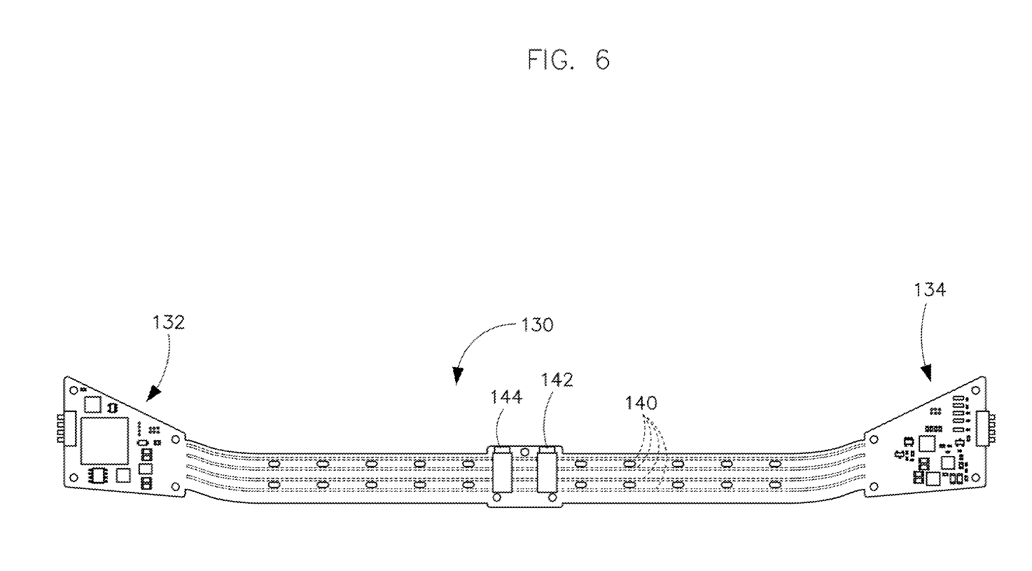

Referring now to FIG. 4-8 a rigid-flex circuit 130 is embedded into the center portion of headband assembly 18. Rigid-flex circuit is an industry term that describes a structure having both rigid and flexible portions, constructed by laminating together rigid and flexible layers and then removing the rigid layers in areas where flexibility is desired. In this application, the term “flex circuit” encompasses rigid-flex circuit, so that rigid-flex circuit is a type of flex circuit. Rigid-flex circuit 130 includes right and left-side rigid portions that support a right-hand electrical network 132, and a left-hand electrical network 134, respectfully. The electrical components of network 132 and 134 are connected together by a first set of conductive traces (not shown) that are internal to rigid-flex circuit 130. These traces are configured in a pattern designed to effect a predetermined scheme of connection. Rigid-flex circuit 130 includes an additional rigid portion, right at the location where the linkage 114 connects to headband assembly 18.

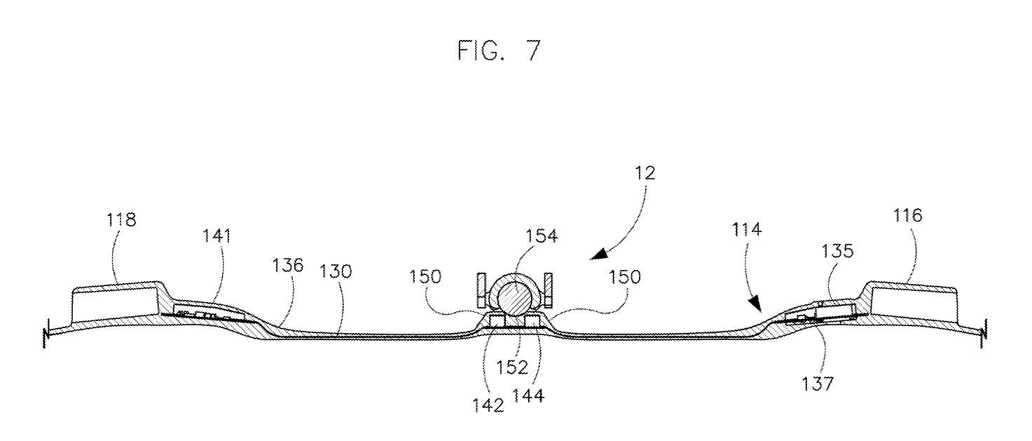

The right-hand network 132 is kept in an air pocket, protected by a right-hand top can 135 (FIG. 7) and a right-hand bottom can 137 (FIG. 7), both made of stainless steel that is 0.15 mm thick. The top can 135 is 4.5 mm high, whereas can 137 is 1.5 mm high. During the molding process, these cans 135 and 137 prevent the polymer material from contacting the components of network 132. Although bottom can 137 does create an area of some rigidity to the outside of strap assembly 18, it is covered by a 0.3 mm thick covering of relatively soft polymeric material 136, which greatly ameliorates this condition. A round indent (not shown) in can 137, which defines a hole (not shown) at its center, provides a seat for the head of a shaft (not shown) for the brightness control knob 124. On the left-hand side, only a top can 141 (FIG. 7), having similar dimensions to and made of the same material as the top right-hand top can 135, is required, due to a smaller component set, confined to the top of rigid-flex circuit 130. In an alternative preferred embodiment (not shown) a type of polymer that permits heat flow is used, thereby eliminating the need cans 135, 137 and 141. In a preferred embodiment (FIG. 1), video processor 24 and microcontroller 46 (in the form of an integrated circuit, in some embodiments capable of substantial data processing) are placed on the right side, as part of network 132, in can 135 to more directly receive input from the brightness control knob. In an alternative preferred embodiment, these elements are placed on the left side, as part of network 134, in can 141. In alternative preferred embodiments, one or more of the cans are larger than described here or are made of a material other than stainless steel, such as titanium.

Electrical networks 132 and 134 are electrically connected together, to bezel 12 and to video camera 14, by a second set of conductive traces 140, each of which extends either across the center of rigid-flex circuit 130 or from one of the electrical networks 132 and 134 to either a first jack 142 or a second jack 144. In a preferred embodiment first jack 142 accepts a plug 146 (FIG. 3) that through wire 148, supplies bezel 12 with electric power. When not in use for this purpose, jack 142 accepts a plug (not shown) from a voltage source, for recharging batteries 32. Second jack 144 accepts a plug 145 (FIG. 2) for the multi-conductor cable 16, that forms a portion of data bus 46 from video camera 14 to processing network 132 or 134. Although plug 145 is shown in the form of an audio plug, which may have several contacts, multi-pin forms are used in alternative preferred embodiments. Plug 146 and the wire attached to it may be considered an electrically conductive system of linkage 114, whereas first jack 142 may be considered a further electrically conductive element of headband assembly 18. Bezel 12 could be electrically connected to headband assembly 18 by a simple wire, in which case the portion of the wire in the linkage could still be considered an electrically conductive system and the portion in the headband could be considered a further electrically conductive element. Video camera 14 must be connected by a set of conductors because of the volume of data required to be moved.

In an alternative preferred embodiment, rigid-flex circuit 130 is replaced by a longitudinal flex circuit or a longitudinal rigid-flex circuit having a circuit board electrically and physically connected to either end, a right-hand circuit board supporting and electrically connecting network 132 and a left had circuit board supporting and electrically connecting network 134. In alternative preferred embodiments the pair of circuit boards is connected by a cable harness or a ribbon cable.

In a preferred embodiment, rigid-flex circuit 130 (together with jacks 142 and 144 and networks 132 and 134) is encased in a sheathing of polymer material 136 that also forms the top arms 126 and side arms 128. To produce the headband assembly 18, rigid-flex circuit 130 is suspended in a mold by shafts that extend through apertures for battery charge indicator lights 121. Polymer material in liquid phase is forced into the mold and after it has been allowed to cure, the shafts are withdrawn and the headband assembly 18 is ejected.

In a preferred embodiment sheathing polymer material 136 may be Styrene-Ethylene/Butylene-Styrene Block Copolymer or similar material, preferably having a shore durometer rating of between 50 and 60 in its cured state. In one preferred embodiment, the shore durometer rating is 55. The 100% modulus is preferably between 1800 and 2500 psi. The mold injection temperature is between 180° C. and 240° C. These materials are available from United Soft Plastics of Lawrenceville, Ga. In one preferred embodiment, an antimicrobial agent is added to the polymer material 136, to prevent fungal and bacterial growth on the surfaces of the material 136, in use. In a preferred embodiment, MCX 122656 Antimicrobial Masterbatch available from RTP Co., of Winona, Minn., which maintains a website at www.rtpcompany.com, is added to the polymeric substance, in liquid state.

In prior art of battery bearing headbands, the battery sockets have been separated from the material contacting the user's head by a space for circuitry, whereas in the preferred embodiment, the circuitry has been placed in front of the battery, as opposed to a position interposed between the battery and the head. Also, the battery sockets 118 have been moved farther back on the head, relative to prior art headbands, so that the closest portion of the batteries 32 to the linkage is 153 mm from the linkage as measured along the headband as it curves about the head, or stated in a slightly different but equivalent manner, measured as it would be if the headband assembly were laid out flat. For most wearers, this places the forwardmost part of the batteries at a position just above the ears, so that a portion of batteries may extend in backward direction at the place where the head curves inwardly toward the back, thereby avoiding contact between the batteries and the head, and providing a greater balance in weight, yielding greater comfort.

There are a number of advantages to the resulting headband. First, as it is constructed as a unitary piece, there are no seams that in other systems provide a foothold for the growth of fungus, and seepage of users' cleaning fluid into interior cavities, which can potentially damage electrical networks 132 and 134. Also, in one prior art system the two pieces that were joined to form the band for the back of the head also formed the panels separating the batteries from the head. This piece was made of a harder polymer material than other portions of the headband, in part to resist the tendency of the batteries, which extended further from the head because of the interposed electrical network, to torque with the top being pulled by gravity downwardly, which could easily translate to away from the head. The use of a harder polymer, however, can result in discomfort over the hours required to complete some surgeries. In headband 18, the use over the entire assembly of polymer material 136 which in a preferred embodiment has a shore durometer reading of 55 is more comfortable, even over long periods of time. In addition, the traces 140 that link networks 132 and 134 permit communication that permits these networks to cooperate. In one preferred embodiment, the battery delivering power to the bezel 12 shifts periodically, for example as the voltage of the active battery passes below a threshold, the load of the optical assembly is shifted to the other battery 32, so that the batteries drain at the same rate, over time. Also, those traces leading from networks 132 and 134 to the jack for supplying bezel 12, and to the data port of camera 14, make external wires unnecessary. Such wires can present a snagging hazard.

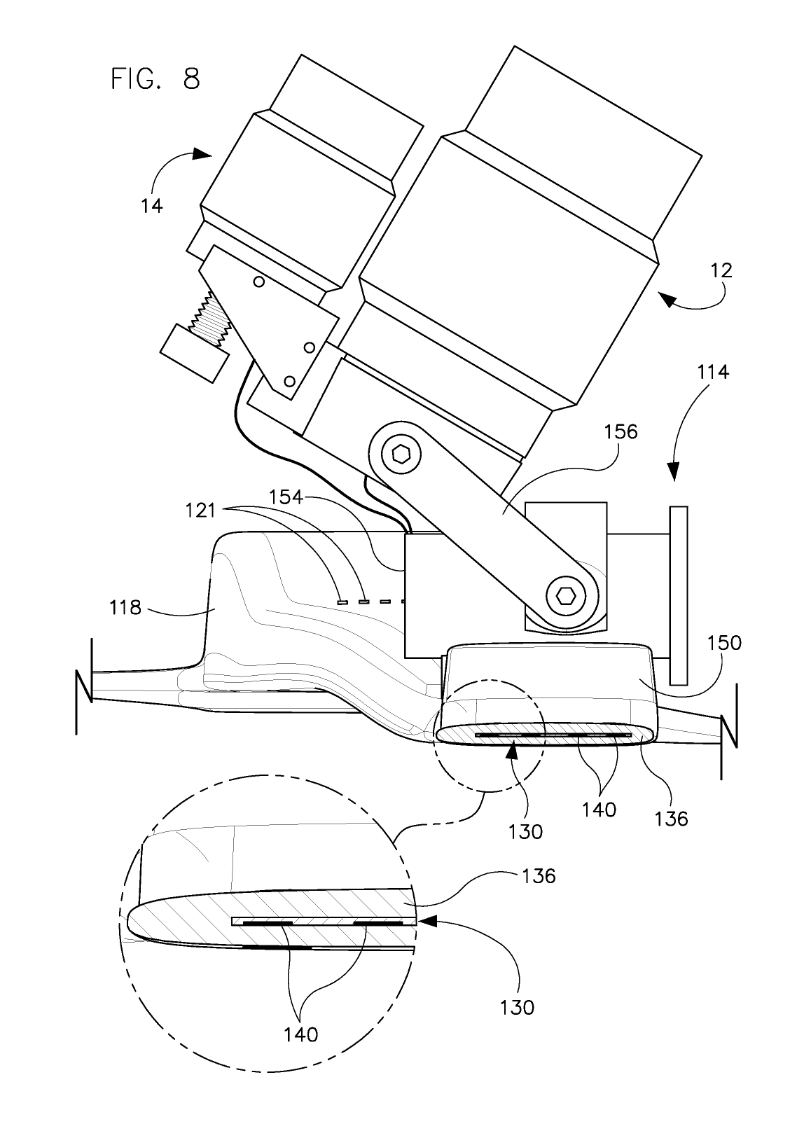

A pair of parallel front-center vertical ridges 150 (FIGS. 7 and 8) are created by the encasement of jacks 142 and 144. The valley 152 between these ridges form an elongated seat for post 154, which is part of support linkage 114. When arms 156 (also part of linkage 114) are rotated, post 154 is torqued and in turn torques headband assembly 18. The structure of post 154 and ridges 150, however, help to diffuse this torque and material 136 helps to cushion the forehead from the torque, so that the operation of rotating arms 156 is not as uncomfortable to the wearer of headlamp and video camera system 10 as it would otherwise be. Linkage 114 includes a partial collar 157, which fits onto post 154. Moreover, partial collar 157 is removable from post 154, so that assembly 115, including headlamp 12 and camera 14, may be quickly removed from headband assembly 18, with plugs 145 and 146 pulled from jacks 142 and 144. Accordingly, if an electrical or mechanical problem is detected with either headlamp 12 or camera 14 the entire unit 115 may be quickly replaced with a spare. Alternatively, a first version of unit 115 can be quickly swapped out for a second version of unit 115, having specialized or more advanced properties. In embodiments, there is an adjust element, such as the threaded bolt shown with camera 14, for adjusting the angle of the camera 14, relative to headlamp 12.

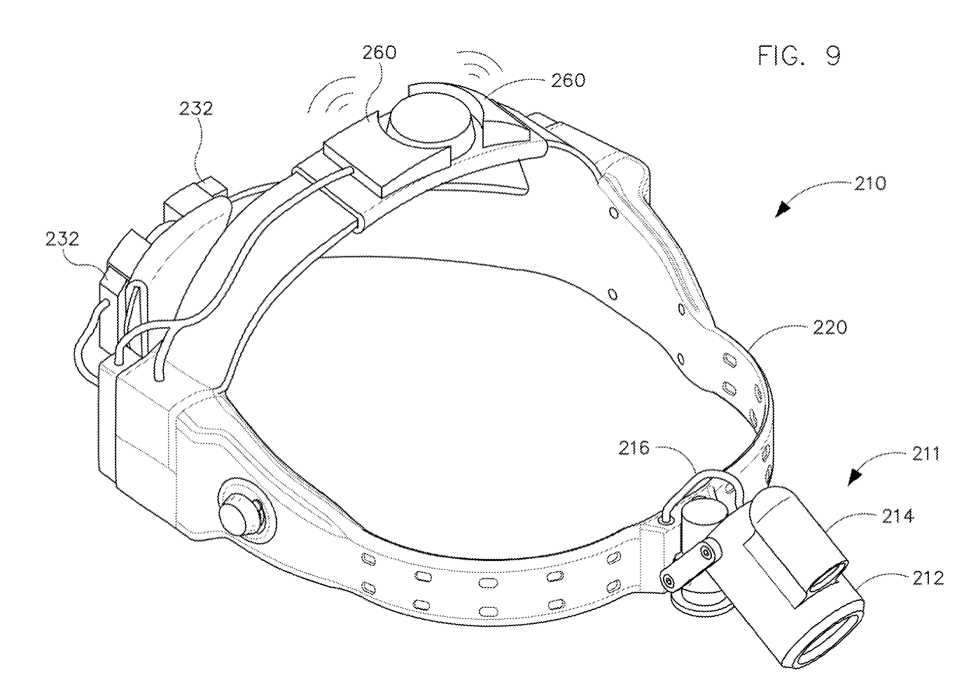

Referring to FIG. 9, an alternative preferred embodiment of a medical headlamp assembly 210 includes a headlamp and camera unit 211, which includes a headlamp 212 and an image sensor module 214 joined together. A single cable 216 carries both electric power to unit 211, where it is divided interior to the housing between headlamp 212 and image sensor 214, and also carries data to and from sensor 214. A pair of antennae 260, driven by wire 262, are located on top of assembly 210 to have a greater chance of having an unblocked line-of-sight to a receiver, such as tablet computer 26. An additional pair of batteries are held at the back, in much the same manner as batteries 32. Assemblies 10 and 210 may be otherwise the same. In the embodiment of FIG. 9, special design features draw heat from the headlamp 212, away from the camera 214, to avoid overheating camera 214.

An issue encountered when integrating a headlamp 12 and an image sensor 14 is heat from the headlamp 12 interfering with the image sensor 14, as image sensors tend to be heat sensitive. One approach to addressing this concern is the use of a configuration like that shown in FIG. 8, where the image sensor 14 and the headlamp have an air gap in between, over most of their surfaces, with some joining bands, preferably made of a heat resistive substance, such as silicone. In the embodiment of FIG. 9, a layer of silicone is interposed between the headlamp 12 and the image sensor 14, within the housing. In one embodiment, a layer of silicone is interposed with the side on the headlamp covered with heat reflective and conductive material, such as copper, so that heat is drawn away from the camera, along the heat conductive layer.

The ability to wirelessly broadcast the video signal of a surgery greatly eases the task of the teaching surgeon, who wishes to bring the student into the operating theater with him, without being tethered by a wire to receiving device or having an additional person actual in the operating room, with him. The greatly facilitated capability of creating a video display of the surgical procedure can be expected to enhance medical education, with the attendant result of better trained junior surgeons, performing their operations in a more expert manner.

While a number of exemplary aspects and embodiments have been discussed above, those possessed of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A medical headlamp and camera system, comprising:
(a) an articulated linkage-and-headlamp assembly, including an articulated linkage supporting a headlamp;
(b) a video camera supported by said articulated linkage-and-headlamp assembly, and which produces a first video data signal;
(c) a headband assembly, supporting said articulated linkage-and-headlamp assembly, and including at least one battery port, supporting a battery, and further including an electrical network, including a microcontroller, that supplies electrical power to said headlamp, in reliance on computations performed by the microcontroller, from said battery and also delivers electrical power from said batteries to said video camera;
(d) a data compression network, electrically connected to said video camera, which receives said first video data signal and compresses it into a compressed data signal; and
(e) a wireless transceiver supported by said headband assembly and electrically connected to said data compression network to wirelessly transmit said compressed data signal.

2. The medical headlamp and camera system of claim 1, wherein said electrical network controls said headlamp responsive in part to said data representative of said first video data signal.

3. The medical headlamp and camera system of claim 1, wherein said integrated circuit processes data representative of said first data signal by detecting a region illuminated by said bezel and eliminating from further processing pixels outside of said illuminated region.

4. The medical headlamp and camera system of claim 1, wherein said data compression network is controlled by said microcontroller.

5. The medical headlamp and camera system of claim 1, wherein said integrated circuit decreases power to said headlamp, when data representative of said first video signal fits a set of criteria.

6. The medical headlamp and camera system of claim 1, wherein said system includes a single switch, which activates said headlamp, and places said wireless transceiver into a listen state, to receive a signal.

7. The medical headlamp and camera system of claim 6, wherein said single switch is a knob, wherein one position of rotation, switches said system into an “off” state.

8. A medical headlamp and camera system, comprising:
(a) a headband assembly, including:
(i) a headband, including at least one battery port, supporting a battery:
(ii) an articulated linkage-and-headlamp assembly supported by said headband, and including an articulated linkage supporting a headlamp and an electrically conductive connection from said electrical network to said headlamp powering said headlamp from said electrical network;
(iii) an electrical network, including a microcontroller, in said headband, receiving electrical power from said battery and delivering electrical power to said electrically conductive connection, thereby powering said headlamp;
(b) an image sensor supported by said headband assembly and electrically and communicatively connected to and powered by said electrical network and producing a first video data signal and delivering said first video signal to said electrical network;
(c) a wireless transceiver supported by said headband assembly and electrically connected to said data compression network to wirelessly transmit said compressed data signal; and
(d) wherein said electrical network processes data representative of said first data signal by detecting a region illuminated by said bezel and eliminating from further processing pixels outside of said illuminated region.

9. The medical headlamp and camera system of claim 8, wherein said camera is supported by said articulated linkage-and-headlamp assembly.

10. The medical headlamp and camera system of claim 8, wherein after said headlamp is activated, an initial determination is made, from an early frame, of which pixels are illuminated by reflection from a spot of light created by said headlamp and this determination is used to eliminate pixels from outside said spot of light in subsequent frames.

11. The medical headlamp and camera system of claim 10, wherein information from subsequent frames is used to refine said initial determination.

12. The medical headlamp and camera system of claim 10, wherein after said initial assessment is made, an operation is periodically performed in which a frame of data is used to update said initial determination.

13. The medical headlamp and camera system of claim 10, wherein said operation is performed every second.

14. A medical headlamp and camera system, comprising:
(a) a headband assembly, including:
(i) a headband, including at least one battery port, supporting a battery, and further including an electrical network, including an integrated circuit;
(ii) an articulated linkage-and-headlamp assembly supported by said headband, and including an articulated linkage supporting a headlamp and an electrically conductive connection from said electrical network to said headlamp powering said headlamp from said electrical network;
(b) an image sensor supported by said headband assembly such that it can be directed to gather imagery from a region illuminated by said headlamp and electrically and communicatively connected to and powered by said electrical network and producing a first video data signal;
(c) a data compression network, electrically connected to said video camera, which receives said first video data signal and compresses it into a compressed data signal;
(d) a wireless transceiver supported by said headband assembly and electrically connected to said data compression network to wirelessly transmit said compressed data signal; and
(e) wherein said electrical network processes data representative of said first data signal and controls said headlamp brightness in response thereto.

15. The medical headlamp and camera system of claim 14, wherein said camera is supported by said articulated linkage-and-headlamp assembly.

16. The medical headlamp and camera system of claim 12, wherein said microcontroller detects pixels from said region illuminated by said headlamp from said first video data signal.

17. The medical headlamp and camera system of claim 14, wherein if said first video data signal shows that any region illuminated by said headlamp is returning light at an above threshold level, said electrical network reduces said headlamp brightness.

18. The medical headlamp and camera system of claim 14, wherein said electrical network determines the average level of light return from said area illuminated by said headlamp, and controls said brightness of said headlamp to achieve a set point average brightness, by increasing said brightness if said average brightness is below said set point, and decreasing said brightness if said average brightness is above said set point.

19. The medical headlamp and camera system of claim 18, wherein said set point is user adjustable.

20. The medical headlamp and camera system of claim 18, wherein said adjustments can be overridden, to achieve a constant light output level from said headlamp.
